(19) United States
(12) Patent Application Publication
(10) Pub. No.: US 2017/0172902 A1
(43) Pub. Date: Jun. 22, 2017

(54) AGENT FOR TEMPORARILY RESHAPING KERATIN-CONTAINING FIBERS COMPRISING PRESERVATIVE

(71) Applicant: Henkel AG & Co. KGaA, Duesseldorf (DE)

(72) Inventors: Anna Puls, Winsen (Luhe) (DE); Marcus Noll, Norderstedt (DE); Arne Junge, Hamburg (DE); Sandra Fuchs, Pinneberg (DE); Nora Koopmann, Hamburg (DE)

(73) Assignee: Henkel AG & Co. KGaA, Duesseldorf (DE)

(21) Appl. No.: 15/377,298

(22) Filed: Dec. 13, 2016

(30) Foreign Application Priority Data
Dec. 17, 2015 (DE) 10 2015 225 560.3

(51) Int. Cl. A61K 8/92; A61K 8/34; A61K 8/81; A61Q 5/06

(52) U.S. Cl. CPC A61K 8/92; A61Q 5/06; A61K 8/345; A61K 8/8176; A61K 8/347; A61K 8/927; A61K 8/922

(57) ABSTRACT

A shelf-stable cosmetic agent for temporarily shaping keratin fibers, and in particular human hair, having improved protection against the fading of the color of keratin fibers is provided.

### CROSS-REFERENCE TO RELATED APPLICATION

This application claims priority to German Patent Application No. 10 2015 225 560.3, filed Dec. 17, 2015, which is incorporated herein by reference in its entirety.

### TECHNICAL FIELD

The present disclosure relates to a cosmetic agent for temporarily shaping keratin fibers, and in particular human hair.

### BACKGROUND

Temporarily creating hair styles for an extended period of up to several days generally requires the use of setting active ingredients. Hair treatment agents that are used to temporarily impart shape to the hair therefore play an important role. Corresponding agents for temporary reshaping usually comprise synthetic polymers and/or waxes serving as the setting active ingredient. Agents for supporting the temporary shaping of hair can be formulated in the form of hair spray, hair wax, hair gel, or hair foam, for example.

The most important property of an agent for temporarily reshaping hair, hereafter also referred to as a styling agent, is to give the treated fibers the strongest hold possible in the newly modeled shape, which is to say a shape that has been imparted to the hair. This is also referred to as strong styling hold or a high degree of hold of the styling agent. The styling hold is essentially determined by the nature and amount of the setting active ingredient that is used, although further components of the styling agent may also have an influence.

In addition to a high degree of hold, styling agents must satisfy a whole host of additional requirements. These can be broken down in approximate terms into properties of the hair, properties of the individual formulation, such as properties of the foam, of the gel, or of the sprayed aerosol, and properties that relate to the handling of the styling agent, wherein the properties of the hair are particularly important. In particular moisture resistance, low tack, and a balanced conditioning effect shall be mentioned. Moreover, a styling agent should be universally suitable for all hair types to an extent as great as possible, and be gentle on the hair and skin.

In order to meet the diverse requirements, a number of synthetic polymers have already been developed as setting active ingredients, which are used in styling agents. The polymers can be divided into cationic, anionic, non-ionic and amphoteric setting polymers. As an alternative or in addition, waxes are used as setting active ingredients. Ideally, the polymers and/or waxes form a polymer film when applied to the hair, or a film that gives the hair style a strong hold on the one hand, but on the other hand is sufficiently flexible so as not to break under stress.

In some instances, however, the use of such styling products can cause the hair color to fade, in particular in the case of dyed hair.

Styling products, in particular those that are present in the form of emulsions, can moreover have instabilities in the form of synereses, which have the undesirable effect of resulting in a short shelf life.

### BRIEF SUMMARY

Cosmetic agents for temporarily shaping keratin fibers and methods for temporarily shaping keratin fibers are provided. In accordance with an exemplary embodiment, a cosmetic agent for temporarily shaping keratin fibers comprises: (a) at least one wax having a melting point above about 37° C. in a total amount of about 1 to about 85 wt. %; (b) at least one emulsifier in a total amount of about 1 to about 30 wt. %; (c) at least one polyhydric alcohol in a total amount of about 0.5 to about 25 wt. %; (d) o-phenylphenol and/or salt(s) of o-phenylphenol in a total amount of about 0.01 to about 0.25 wt. %; and (e) water in a total amount of about 0.5 to about 90 wt. %, wherein the weight information is based in each case on the total weight of the cosmetic agent.

In accordance with another exemplary embodiment, a method for temporarily shaping keratin fibers comprises providing a cosmetic agent comprising: (a) at least one wax having a melting point above about 37° C. in a total amount of about 1 to about 85 wt. %; (b) at least one emulsifier in a total amount of about 1 to about 30 wt. %; (c) at least one polyhydric alcohol in a total amount of about 0.5 to about 25 wt. %; (d) o-phenylphenol and/or salt(s) of o-phenylphenol in a total amount of about 0.01 to about 0.25 wt. %; and (e) water in a total amount of about 0.5 to about 90 wt. %, wherein the weight information is based in each case on the total weight of the cosmetic agent. The method further comprises applying the cosmetic agent to the keratin fibers.

### DETAILED DESCRIPTION

The following detailed description is merely exemplary in nature and is not intended to limit the invention or the application and uses of the invention. Furthermore, there is no intention to be bound by any theory presented in the preceding background or the following detailed description.

Stable, and in particular shelf-stable, wax-containing cosmetic agents for temporarily shaping keratin fibers, which do not cause any, or reduced, fading of the color of keratin fibers, in particular of human hair, are provided herein.

In this regard, a cosmetic agent for temporarily shaping keratin fibers comprises:

(a) at least one wax having a melting point above about 37° C. in a total amount of about 1 to about 85 wt. %;
(b) at least one emulsifier in a total amount of about 1 to about 30 wt. %;
(c) at least one polyhydric alcohol in a total amount of about 0.5 to about 25 wt. %;
(d) o-phenylphenol and/or salt(s) of o-phenylphenol in a total amount of about 0.01 to about 0.25 wt. %; and
(e) water in a total amount of about 0.5 to about 90 wt. %,
wherein the weight information is based in each case on the total weight of the cosmetic agent.

Cosmetic agents for temporarily shaping human hair are also referred to as styling agents. The present disclosure relates in particular to styling agents such as hair waxes, pastes, lotions or clays. The product form “clay” refers to high viscosity, wax-like cosmetic agents containing clay compounds (such as kaolin), among other things.

Surprisingly, it was found within the scope contemplated herein that adding o-phenylphenol to a cosmetic agent for temporarily reshaping keratin fibers, and in particular human hair, allowed fading of the color of the fibers, and in particular fading of the color of dyed human hair, to be reduced when using the cosmetic agents.

Surprisingly, it was also found that the physical stability of cosmetic agents in the form of emulsions was able to be increased, and these agents exhibit no phase separation (syneresis).

Moreover, the microbiological stability of the cosmetic agents was increased.

Other properties that are usually required of cosmetic agents for temporarily shaping keratin fibers such as long-term hold, stiffness and low tack are preserved.

As contemplated herein, the term “keratin fibers” comprises furs, wool and feathers, but in particular human hair.

The cosmetic agent comprises at least one natural or synthetic wax having a melting point of above about 37° C. as component (a). The cosmetic agent comprises the at least one wax in a total amount of about 1 to about 85 wt. %, preferably about 1.5 to about 50 wt. %, more preferably about 2 to about 30 wt. %, and still more preferably about 5 to about 25 wt. %, based on the total weight of the cosmetic agent.

Natural or synthetic waxes that can be used include solid paraffins or isoparaffins, plant-based waxes such as candelilla wax, carnauba wax, esparto grass wax, Japan wax, cork wax, sugar cane wax, ouricury wax, montan wax, sunflower wax, fruit waxes, and animal waxes, such as beeswaxes and other insect waxes, cetaceum, shellac wax, wool fat and rump fat, furthermore mineral waxes such as ceresin and ozokerite, or petrochemical waxes, such as petrolatum, paraffin waxes, microwaxes made of polyethylene or polypropylene, and polyethylene glycol waxes. It may be advantageous to use hydrogenated waxes. Furthermore, it is also possible to use chemically modified waxes, in particular the hard waxes, such as montan ester waxes, sasol waxes and hydrogenated jojoba waxes.

Also suitable are the triglycerides of saturated and optionally hydroxylated C16-30 fatty acids, such as hydrogenated triglyceride fats (hydrogenated palm oil, hydrogenated coconut oil, hydrogenated castor oil), glyceryl tribehenate or glyceryltri-12-hydroxy stearate, furthermore synthetic full esters of fatty acids and glycols (such as Syncrowachs®) or polyols having 2 to 6 carbon atoms, fatty acid monoalkanol amides including a C12-22 acyl group and a C2-4 alkanol group, esters of saturated and/or unsaturated, branched and/or unbranched alkane carboxylic acids having a chain length of 1 to 80 carbon atoms and saturated and/or unsaturated, branched and/or unbranched alcohols having a chain length of 1 to 80 carbon atoms, including, for example, synthetic fatty acid/fatty alcohol esters such as stearyl stearate or cetyl palmitate, esters of aromatic carboxylic acids, dicarboxylic acids or hydroxycarboxylic acids (such as 12-hydroxystearic acid), and saturated and/or unsaturated, branched and/or unbranched alcohols having a chain length of 1 to 80 carbon atoms, lactides of long-chain hydroxycarboxylic acids, and full esters of fatty alcohols and dicarboxylic and tricarboxylic acids, such as dicetyl succinate or dicetyl/distearyl adipate, and mixtures of these substances.

The wax components can also be selected from the group of the esters of saturated, unbranched alkane carboxylic acids having a chain length of 14 to 44 carbon atoms and saturated, unbranched alcohols having a chain length of 14 to 44 carbon atoms, provided the wax component or the collectivity of the wax components is solid at room temperature. The wax components can be selected, for example, from the group consisting of the C16-36 alkyl stearates, the C10-40 alkyl stearates, the C2-40 alkyl isostearates, the C20-40 dialkyl esters of dimer acids, the C18-38 alkyl hydroxystearoyl stearates, the C20-40 alkyl erucates, and furthermore C30-50 alkyl beeswax and cetearyl behenate can be used. Silicone waxes, such as stearyl trimethylsilane/stearyl alcohol, are also optionally advantageous. Preferred wax components are the esters of saturated, monohydric C20 to C60 alcohols and saturated C8 to C30 monocarboxylic acids, preferably in particular a C20 to C40 alkyl stearate, which is available from Koster Keunen Inc. by the name Kesterwachs® K82H.

Natural, chemically modified and synthetic waxes can be used alone or in combination. The teaching contemplated herein thus also comprises the combined use of multiple waxes. Furthermore, a number of wax mixtures, optionally blended with further additives, is commercially available. Examples of mixtures that can be used include those by the designations “Spezialwachs 7686 OE” (mixture of cetyl palmitate, beeswax, microcrystalline wax and polyethylene having a melting point of 73 to 75° C.; manufacturer: Kahl & Co), Polywax® GP 200 (a mixture of stearyl alcohol and polyethylene glycol stearate having a melting point of 47 to 51° C.; manufacturer: Croda) and “Weichceresin® FL 400” (a paraffin jelly/liquid paraffin/wax mixture having a melting point of 50 to 54° C.; manufacturer: Parafluid Mineralölgesellschaft).

The wax (a) is particularly preferably selected from plant-based waxes, in particular carnauba wax (INCI: Copernicia Cerifera Cera), beeswax (INCI: Beeswax Cera Alba), Petrolatum (INCI), microcrystalline wax, and in particular mixtures thereof.

Preferred mixtures comprise the combination of carnauba wax (INCI: Copernicia Cerifera Cera), petrolatum, and microcrystalline wax.

The wax or the wax components should be solid at about 25° C. and should melt around >37° C. In preferred embodiments contemplated herein, about 50 wt. % or more of the entire wax fraction (a), and preferably about 70 wt. % to about 90 wt. % of the entire wax fraction (a), has a melting point in the range of about 45 to about 60° C.

The agent comprises at least one emulsifier as the further essential component (b). In principle, anionic, cationic, non-ionic and ampholytic surface-active compounds which are suitable for use on the human body can be used as emulsifiers. The ampholytic surface-active compounds comprise zwitterionic surface-active compounds and ampholytes. Non-ionic emulsifiers are preferred.

Non-ionic emulsifiers that can be used include in particular addition products of ethylene oxide to linear fatty alcohols, to fatty acids, to fatty acid alkanolamides, to fatty acid monoglycerides, to sorbitan fatty acid monoesters, to fatty acid glycerides, to methyl glucoside monofatty acid esters, to polydimethyl siloxanes, and mixtures thereof.

The at least one emulsifier (b) is preferably selected from non-ionic emulsifiers such as addition products of about 2 to about 50 moles ethylene oxide to linear fatty alcohols having 8 to 30, preferably 12 to 18 carbon atoms, addition products of about 2 to about 50 moles ethylene oxide and about 1 to about 5 moles propylene oxide to linear fatty alcohols having 8 to 30, preferably 12 to 18 carbon atoms, addition products of about 2 to about 100 moles ethylene oxide to linear fatty acids having 12 to 18 carbon atoms, and mixtures thereof.

Examples of particularly preferred non-ionic surfactants are compounds having the INCI names Steareth-2, Steareth-21, Oleth-10, PEG-100 Stearate or PPG-5-Ceteth-20, and in particular combinations thereof.

Likewise preferred emulsifiers are the esters of fatty acids having 12 to 18 carbon atoms with saccharides. In particular the monoesters and/or diesters of sucrose with stearic acid and/or palmitic acids are preferably used. Examples of particularly preferred non-ionic emulsifiers are compounds having the INCI names Sucrose Stearate, Sucrose Distearate, and mixtures thereof.

Further preferred emulsifiers are linear fatty acids having 12 to 22 carbon atoms and mixtures thereof. The linear fatty acids can be present in neutralized and/or non-neutralized form, depending on the pH value.

Likewise preferred emulsifiers (b) are addition products of about 2 to about 20 moles ethylene oxide to beeswax, such as in particular the compounds having the INCI names PEG-6 Beeswax, PEG-8 Beeswax, PEG-12 Beeswax or PEG-20 Beeswax. PEG-8 Beeswax is particularly preferred from this class of emulsifiers.

Another class of emulsifiers (b) that can preferably be used is the monoesters of fatty acids having 12 to 22 carbon atoms with glycerol. In particular the monoesters of glycerol with stearic acid and/or palmitic acids are preferably used. Examples of particularly preferred emulsifiers are compounds having the INCI names Glyceryl Stearate, Glyceryl Palmitate, or mixtures thereof.

Phosphate surfactants are likewise a class that can be used as emulsifiers (b).

Phosphate surfactants are compounds that, in addition to at least one hydrophobic molecule part, which is usually long-chain alkyl groups, comprise a hydrophilic part, which comprises a phosphate group and optionally polyethylene oxide and/or polypropylene units.

Preferably, C₈ to C₃₀ alkyl groups, and particularly preferably C₁₂ to C₂₄ alkyl groups, are used as the hydrophobic part, which may be saturated or unsaturated, and possibly may be branched. Depending on origin and production, it is preferred to use a mixture of compounds having differing alkyl chain lengths as the phosphate surfactant.

The phosphate surfactants can be used either in the protonated form thereof, which is to say in the acid form, or in the form of corresponding phosphate salts, wherein alkali metal ions, ammonium ions, mono-, di- or tri-alkylammonium ions, and mono-, di- or tri-(hydroxyalkyl)ammonium ions are preferred suitable counterions. Particularly preferred phosphate surfactants are used as free acids or as sodium salt, ammonium salt, ethanolamine salt (MEA), diethanolamine salt (DEA), or triethanolamine salt (TEA).

The hydrophilic part of the phosphate surfactant can optionally comprise polyethylene oxide and/or polypropylene units, in addition to the phosphate group. Phosphate surfactants that comprise 1 to 15, and preferably 3 to 10, ethylene oxide units and/or 1 to 10, and preferably 3 to 8, propylene oxide units are preferred.

It is preferred for the emulsifier (b) to comprise at least one phosphate surfactant selected from C₈-C₃₀ alkyl phosphate, C₈-C₃₀ alkyl ether phosphate, di(C₈-C₃₀-alkyl) phosphate and/or mixtures thereof, preferably selected from C₁₂-C₂₄ alkyl phosphate, C₁₂-C₂₄ alkyl ether phosphate, di(C₁₂-C₂₄-alkyl) phosphate and/or mixtures thereof.

Examples of suitable C₈-C₃₀ alkyl phosphates are decyl phosphates, lauryl phosphates, C₁₂-C₁₃ alkyl phosphates, myristyl phosphates, cetyl phosphates, stearyl phosphates, isostearyl phosphates, cetearyl phosphates, oleyl phosphates, and behenyl phosphates.

Examples of suitable C₈-C₃₀ alkyl ether phosphates are Deceth-3 phosphates, Deceth-4 phosphates, Deceth-5 phosphates, Deceth-8 phosphates, Deceth-10 phosphates, Deceth-12 phosphates, Deceth-15 phosphates, Laureth-3 phosphates, Laureth-4 phosphates, Laureth-5 phosphates, Laureth-8 phosphates, Laureth-10 phosphates, Laureth-12 phosphates, Laureth-15 phosphates, Ceteth-3 phosphates, Ceteth-4 phosphates, Ceteth-5 phosphates, Ceteth-8 phosphates, Ceteth-10 phosphates, Ceteth-12 phosphates, Ceteth-15 phosphates, Steareth-3 phosphates, Steareth-4 phosphates, Steareth-5 phosphates, Steareth-8 phosphates, Steareth-10 phosphates, Steareth-12 phosphates, Steareth-15 phosphates, Ceteareth-3 phosphates, Ceteareth-4 phosphates, Ceteareth-5 phosphates, Ceteareth-8 phosphates, Ceteareth-10 phosphates, Ceteareth-12 phosphates, Ceteareth-15 phosphates, Oleth-3 phosphates, Oleth-4 phosphates, Oleth-5 phosphates, Oleth-8 phosphates, Oleth-10 phosphates, Oleth-12 phosphates, Oleth-15 phosphates, as well as PPG-3-Ceteth-10 phosphates, PPG-3-Ceteth-15 phosphates, PPG-5-Ceteth-10 phosphates, PPG-5-Ceteth-15 phosphates, PPG-3-Oleth-10 phosphates, PPG-3-Oleth-15 phosphates, PPG-5-Oleth-10 phosphates, and PPG-5-Oleth-15 phosphates.

Examples of suitable di(C₈-C₃₀ alkyl) phosphates are didecyl phosphates, dilauryl phosphates, di(C₁₂-C₁₃ alkyl) phosphates, dimyristyl phosphates, dicetyl phosphates, distearyl phosphates, diisostearyl phosphates, dicetearyl phosphates, dioleyl phosphates, and dibehenyl phosphates.

In a preferred embodiment, the emulsifier (b) is selected from the group consisting of addition products of about 2 to about 100 moles ethylene oxide to linear fatty acids having 12 to 18 carbon atoms, monoesters of fatty acids having 12 to 18 carbon atoms with glycerol, addition products of about 2 to about 20 moles ethylene oxide to beeswax, phosphate surfactants, and mixtures thereof.

The cosmetic agent comprises the at least one emulsifier in a total amount of about 1 to about 30 wt. %, preferably about 1.5 to about 20 wt. %, and more preferably about 2 to about 20 wt. %, based on the total weight of the cosmetic agent.

The cosmetic agent furthermore comprises a monohydric alcohol as the essential component (c). The amount of monohydric alcohol, based on the total amount of cosmetic agent, is about 0.5 to about 25 wt. %.

Preferred polyhydric alcohols are polyhydric C₂ to C₉ alkanols having two to six hydroxyl groups and polyethylene glycols having 3 to 20 ethylene oxide units. The agents comprise at least one polyhydric C₂ to C₉ alkanol having two to six hydroxyl groups, or at least one water-soluble polyethylene glycol having 3 to 20 ethylene oxide units, or mixtures of at least one polyhydric C₂ to C₉ alkanol having two to six hydroxyl groups and at least one water-soluble polyethylene glycol having 3 to 20 ethylene oxide units.

The C₂ to C₉ alkanols having two to six hydroxyl groups, selected from 1,2-propylene glycol, 1,3-propylene glycol, 2-methyl-1,3-propanediol, glycerol, 1,2-butylene glycol, 1,3-butylene glycol, 1,4-butylene glycol, 1,2-pentanediol, 1,5-pentanediol, 1,2-hexanediol, 1,6-hexanediol, 1,2,6-hexanetriol, 1,2-octanediol, 1,8-octanediol, dipropylene glycol, tripropylene glycol, diglycerol, triglycerol, erythritol, sorbitol, cis-1,4-dimethylol cyclohexane, trans-1,4-dimethylol cyclohexane, arbitrary isomer mixtures of cis- and trans-1,4-dimethylol cyclohexane, and mixtures of these polyhydric alcohols, are preferred. Suitable water-soluble polyethylene glycols are selected from PEG-4, PEG-6, PEG-7, PEG-8, PEG-9, PEG-10, PEG-12, PEG-14, PEG-16, PEG-18 PEG-20, PEG-32, PEG-33, PEG-40, PEG-45, PEG-55, PEG-60, PEG-75, PEG-80, PEG-90, PEG-100, PEG-135, PEG-150, PEG-180, PEG-200, PEG-220, PEG-240, PEG-350, PEG-400, PEG-500, PEG-800, PEG-2M, PEG-5M, PEG-7M, PEG-9M, PEG-14M, PEG-20M, PEG-23M, PEG-25M, PEG-45M, PEG-65M, PEG-90M, PEG-115M, PEG-160M, PEG-180M, and mixtures thereof.

Further suitable polyhydric alcohols are methylol compounds, such as in particular trimethylolethane, trimethylolpropane, trimethylolbutane, pentaerythritol and dipentaerythritol, sugar alcohols having 5 to 12 carbon atoms, such as sorbitol or mannitol, sugar having 5 to 12 carbon atoms, such as glucose or saccharose, or amino sugar, such as glucamine. Dexpanthenol (INCI: Panthenol) is another suitable polyhydric alcohol.

Especially particularly preferred polyhydric alcohols are selected from the group consisting of 1,2-propylene glycol, 1,3-propylene glycol, 1,2-butylene glycol, 1,3-butylene glycol, 1,4-butylene glycol, glycerol, Panthenol (INCI), and mixtures thereof.

Preferred cosmetic agents comprise the polyhydric alcohol(s), based on the weight thereof, in a total amount of about 1 to about 15 wt. %, and more preferably in a total amount of about 2 to about 10 wt. %.

The cosmetic agent furthermore comprises o-phenylphenol and/or salt(s) of o-phenylphenol as the essential component (d). Preferably o-phenylphenol is used, wherein the use within certain narrow quantity ranges is particularly effective.

Preferred cosmetic agents, based on the weight thereof, comprise about 0.01 to about 0.25 wt. %, preferably about 0.05 to about 0.225 wt. %, and more preferably about 0.1 to about 0.2 wt. % o-phenylphenol.

In addition to o-phenylphenol or instead of the same, it is also possible to use salts of o-phenylphenol. For this purpose, among other things the alkali metal salts sodium ortho-phenyl-phenolate, potassium ortho-phenyl-phenolate, as well as ammonium ortho-phenyl-phenolate, magnesium ortho-phenyl-phenolate, calcium ortho-phenyl-phenolate, zinc ortho-phenyl-phenolate, iron ortho-phenyl-phenolate and manganese ortho-phenyl-phenolate have proven to be particularly suitable. Preferred cosmetic agents comprise sodium ortho-phenyl-phenolate.

Cosmetic agents comprise salt(s) from the group consisting of sodium ortho-phenyl-phenolate, potassium ortho-phenyl-phenolate, as well as ammonium ortho-phenyl-phenolate, magnesium ortho-phenyl-phenolate, calcium phenyl-phenolate, zinc ortho-phenyl-phenolate, iron ortho-phenyl-phenolate and manganese ortho-phenyl-phenolate in a total amount of about 0.01 to about 0.25 wt. %, preferably about 0.05 to about 0.225 wt. %, and more preferably about 0.1 to about 0.2 wt. %, in each case based on the weight of the agent.

The cosmetic agent comprises water. Preferred cosmetic agents comprise water as the cosmetic carrier. In these embodiments, the cosmetic agent comprises water as the main component. The water content of the cosmetic agents is about 0.5 to about 90 wt. %, preferably about 15 to about 80 wt. %, and more preferably about 40 to about 75 wt. %, based on the total weight of the cosmetic agent.

The cosmetic agent can furthermore comprise at least one film-forming polymer (f) that is different from the wax component (a). Examples are cationic, anionic, non-ionic or amphoteric polymers. The cosmetic agent can comprise the at least one film-forming polymer (f) in a total amount of about 1 to about 60 wt. %, preferably about 1.5 to about 20 wt. %, and more preferably about 2 to about 15 wt. %, based on the total weight of the cosmetic agent.

Examples include acrylamide/ammonium acrylate copolymer, acrylamides/DMAPA acrylates/methoxy PEG methacrylate copolymer, acrylamidopropyltrimonium chloride/acrylamide copolymer, acrylamidopropyltrimonium chloride/acrylates copolymer, acrylates/acetoacetoxyethyl methacrylate copolymer, acrylates/acrylamide copolymer, acrylates/ammonium methacrylate copolymer, acrylates/t-butylacrylamide copolymer, acrylates copolymer, acrylates/C1-2 succinates/hydroxyacrylates copolymer, acrylates/lauryl acrylate/stearyl acrylate/ethylamine oxide methacrylate copolymer, acrylates/octylacrylamide copolymer, acrylates/octylacrylamide/diphenyl amodimethicone copolymer, acrylates/stearyl acrylate/ethylamine oxide methacrylate copolymer, acrylates/VA copolymer, acrylates/VP copolymer, adipic acid/diethylenetriamine copolymer, adipic acid/dimethylaminohydroxypropyl diethylenetriamine copolymer, adipic acid/epoxypropyl diethylenetriamine copolymer, adipic acid/isophthalic acid/neopentyl glycol/trimethylolpropane copolymer, allyl stearate/VA copolymer, aminoethylacrylate phosphate/acrylates copolymer, aminoethylpropanediol-acrylates/acrylamide copolymer, aminoethylpropanediol-AMPD-acrylates/diacetoneacrylamide copolymer, ammonium VA/acrylates copolymer, AMPD-acrylates/diacetoneacrylamide copolymer, AMP-acrylates/allyl methacrylate copolymer, AMP-acrylates/C1-18 alkyl acrylates/C1-8 alkyl acrylamide copolymer, AMP-acrylates/diacetoneacrylamide copolymer, AMP-acrylates/dimethylaminoethylmethacrylate copolymer, Bacillus/rice bran extract/soybean extract ferment filtrate, bis-butyloxyamodimethicone/PEG-60 copolymer, butyl acrylate/ethylhexyl methacrylate copolymer, butyl acrylate/hydroxypropyl dimethicone acrylate copolymer, butylated PVP, butyl ester of ethylene/MA copolymer, butyl ester of PVM/MA copolymer, calcium/sodium PVM/MA copolymer, corn starch/acrylamide/sodium acrylate copolymer, diethylene glycolamine/epichlorohydrin/piperazine copolymer, dimethicone crosspolymer, diphenyl amodimethicone, ethyl ester of PVM/MA copolymer, hydrolyzed wheat protein/PVP crosspolymer, isobutylene/ethylmaleimide/hydroxyethylmaleimide copolymer, isobutylene/MA copolymer, isobutylmethacrylate/bis-hydroxypropyl dimethicone acrylate copolymer, isopropyl ester of PVM/MA copolymer, lauryl acrylate crosspolymer, lauryl methacrylate/glycol dimethacrylate crosspolymer, MEA-sulfite, methacrylic acid/sodium acrylamidomethyl propane sulfonate copolymer, methacryloyl ethyl betaine/acrylates copolymer, octylacrylamide/acrylates/butylaminoethyl methacrylate copolymer, PEG/PPG-25/25 dimethicone/acrylates copolymer, PEG-8/SMDI copolymer, polyacrylamide, polyacrylate-6, polybeta-alanine/glutaric acid crosspolymer, polybutylene terephthalate, polyester-1, polyethylacrylate, polyethylene terephthalate, polymethacryloyl ethyl betaine, polypentaerythrityl terephthalate, polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, polyvinyl acetate, polyvinyl butyral, polyvinylcaprolactam, polyvinylformamide, polyvinyl imidazolinium acetate, polyvinyl methyl ether, potassium butyl ester of PVM/MA copolymer, potassium ethyl ester of PVM/MA copolymer, PPG-70 polyglyceryl-10 ether, PPG-12/SMDI copolymer, PPG-51/SMDI copolymer, PPG-10 sorbitol, PVM/MA copolymer, PVP, PVP/VA/itaconic acid copolymer, PVP/VA/vinyl propionate copolymer, rhizobian gum, rosin acrylate, shellac, sodium butyl ester of PVM/MA copolymer, sodium ethyl ester of PVM/MA copolymer, sodium polyacrylate, sterculia urens gum, terephthalic acid/isophthalic acid/sodium isophthalic acid sulfonate/glycol copolymer, trimethylolpropane triacrylate, trimethylsiloxysilylcarbamoyl pullulan, VA/crotonates copolymer, VA/crotonates/methacryloxybenzophenone-1 copolymer, VA/crotonates/vinyl neodecanoate copolymer, VA/crotonates/vinyl propionate copolymer, VA/DBM copolymer, VA/vinyl butyl benzoate/crotonates copolymer, vinylamine/vinyl alcohol copolymer, vinyl caprolactam/VP/dimethylaminoethyl methacrylate copolymer, VP/acrylates/lauryl methacrylate copolymer, VP/dimethylaminoethylmethacrylate copolymer, VP/DMAPA acrylates copolymer, VP/hexadecene copolymer, VP/VA Copolymer, VP/vinyl caprolactam/DMAPA acrylates copolymer, yeast palmitate, styrene/VP copolymer, triacontanyl PVP, cellulose ether, such as hydroxypropyl cellulose, hydroxyethyl cellulose and hydroxypropyl methylcellulose, as they are sold under the trademarks Culminal and Benecel (AQUALON), for example.

Furthermore, siloxanes are suitable as film-forming polymers. These siloxanes can either be water-soluble or water-insoluble. Both volatile and non-volatile siloxanes are suitable, wherein non-volatile siloxanes shall be understood to mean those compounds having a boiling point above about 200° C. at normal pressure. Preferred siloxanes are polydialkylsiloxanes, such as polydimethylsiloxane, polyalkylarylsiloxanes, such as polyphenylmethylsiloxane, ethoxylated polydialkylsiloxanes, and polydialkylsiloxanes containing amine and/or hydroxy groups. Glycosidically substituted silicones may also be used.

Homopolyacrylic acid (INCI: Carbomer), which is commercially available in different embodiments under the name Carbopol®, is also a suitable film-forming polymer.

The film-forming polymer is preferably selected from vinylpyrrolidone-containing polymers. The film-forming polymer is particularly preferably selected from the group consisting of polyvinylpyrrolidone, vinylpyrrolidone/vinyl acetate copolymer, Vinyl Caprolactam/VP/Dimethylaminoethyl Methacrylate Copolymer (INCI), VP/DMAPA Acrylates Copolymer (INCI), Triacontanyl PVP (INCI) and mixtures thereof.

Preferred vinylpyrrolidone/vinyl acetate copolymers are vinylpyrrolidone/vinyl acetate copolymers in which the ratio of vinylpyrrolidone (VP) to vinyl acetate (VA) is preferably about 50:70 to about 30:50, and more preferably approximately about 60:40. These are, for example, under the trademark Luviskol (BASF), such as Luviskol VA 64 and Luviskol VA 73.

In particular nourishing components, such as oils, should be mentioned as further suitable auxiliary agents and additives.

Suitable oils are selected from among the esters of the linear or branched, saturated or unsaturated fatty alcohols having 2 to 30 carbon atoms with linear or branched, saturated or unsaturated fatty acids having 2 to 30 carbon atoms, which may be hydroxylated. These include cetyl-2-ethylhexanoate, 2-hexyldecyl stearate (for example, Eutanol® G 16 S), 2-hexyldecyl laurate, isodecyl neopentanoate, isononyl isononanoate, 2-ethylhexyl palmitate (for example, Cegesoft® C 24) and 2-ethylhexyl stearate (for example Cetiol® 868). Likewise preferred are isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid-2-butyl octanoate, diisotridecyl acetate, n-butyl stearate, n-hexyl laurate, n-decyl oleate, oleyl oleate, oleyl erucate, erucyl oleate, erucyl erucate, ethylene glycol dioleate, and ethylene glycol dipalmitate. Cetyl-2-ethylhexanoate is particularly preferred.

Further preferred oils are selected from natural and synthetic hydrocarbons, particularly preferably from mineral oils, paraffin oils, C₁₈ to C₃₀ isoparaffins, in particular isoeicosane, polyisobutene and polydecene, which are available under the designation Emery® 3004, 3006, 3010 or under the designation Ethylflo® from Albemarle or Nexbase® 2004G from Nestle, for example, and further selected from C₈ to C₁₆ isoparaffins, in particular from isodecane, isododecane, isotetradeane and isohexadecane, and mixtures thereof, as well as 1,3-bis(2-ethylhexyl)cyclohexane (available under the trade name Cetiol® S from BASF, for example).

Further preferred oils are selected from the benzoic acid esters of linear or branched C8-22 alkanols. Particularly preferred are benzoic acid-C12-C15-alkyl esters, for example available as the commercial product Finsolv® TN, benzoic acid isostearyl esters, for example available as the commercial product Finsolv® SB, ethylhexyl benzoate, for example available as the commercial product Fiinsolv® EB, and benzoic acid octyldodecyl esters, for example available as the commercial product Finsolv® BOD.

Further preferred oils are selected from fatty alcohols having 6 to 30 carbon atoms, which are unsaturated, or branched and saturated, or branched and unsaturated. The branched alcohols are frequently also referred to as Guerbet alcohols since they can be obtained by way of the Guerbet reaction. Preferred alcohol oils are 2-hexyldecanol (Eutanol® G 16), 2-octyldodecanol (Eutanol® G), 2-ethylhexyl alcohol and isostearyl alcohol.

Further preferred oils are selected from mixtures of Guerbet alcohols and Guerbet alcohol esters, for example the commercial product Cetiol® PGL (2-hexyldecanol and 2-hexyldecyl laurate).

Further preferred cosmetic oils are selected from the triglycerides (=triple esters of glycerol) of linear or branched, saturated or unsaturated, optionally hydroxylated C8-30 fatty acids. The use of natural oils can be particularly preferred, such as amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderberry seed oil, currant seed oil, jojoba oil, linseed oil, macadamia nut oil, corn oil, almond oil, marula oil, evening primrose oil, olive oil, palm oil, palm kernel oil, Brazil nut oil, pecan nut oil, peach kernel oil, rapeseed oil, castor oil, sea buckthorn pulp oil, sea buckthorn kernel oil, sesame oil, soy bean oil, sunflower oil, grape seed oil, walnut oil, wild rose oil, wheat germ oil, and the liquid components of coconut oil, and the like. However, synthetic triglyceride oils, in particular capric/caprylic triglycerides, such as the commercial products Myritol® 318, Myritol® 331 (BASF) or Miglyol® 812 (Hüls) comprising unbranched fatty acid esters and glyceryl triisostearol with branched fatty acid esters are also preferred.

Further preferred cosmetic oils are selected from the dicarboxylic acid esters of linear or branched C2 to C10 alkanols, in particular diisopropyl adipate, di-n-butyl adipate, di-(2-ethylhexyl) adipate, dioctyl adipate, diethyl-/di-n-butyl/dioctyl sebacate, diisopropyl sebacate, dioctyl malate, dioctyl maleate, dicaprylyl maleate, diisooctyl succinate, di-2-ethylhexyl succinate, and di-(2-hexyldecyl) succinate.

Further preferred cosmetics oils are selected from the addition products of 1 to 5 propylene oxide units to monohydric or polyhydric C8-22 alkanols, such as octanol, decanol, decanediol, lauryl alcohol, myristyl alcohol, and stearyl alcohol, for example PPG-2 myristyl ether and PPG-3 myristyl ether (Witconol® APM).

Further preferred cosmetic oils are selected from the addition products of at least 6 ethylene oxide units and/or propylene oxide units to monohydric or polyhydric C3-22 alkanols, such as glycerol, butanol, butanediol, myristyl alcohol and stearyl alcohol, which may optionally be esterified, such as PPG-14 butyl ether (Ucon Fluid® AP), PPG-9 butyl ether (Breox® B25), PPG-10 butanediol (Macol® 57), PPG-15 stearyl ether (Arlamol® E), and glycereth-7-diisononanoate.

Further preferred cosmetic oils are selected from the C8 to C22 fatty alcohol esters of monovalent or polyvalent C2 to C7 hydroxycarboxylic acids, in particular the esters of glycolic acid, lactic acid, malic acid, tartaric acid, citric acid, and salicylic acid. Such esters based on linear C14/15 alkanols, such as C12 to C15 alkyl lactate, and on C12/13 alkanols branched at the 2-position, may be purchased under the trademark Cosmacol® from Nordmann, Rassmann GmbH & Co., Hamburg, in particular the commercial products Cosmacol® ESI, Cosmacol® EMI, and Cosmacol® ETI.

Further preferred cosmetic oils are selected from the symmetric, asymmetric or cyclic esters of carbonic acid with C₃₋₂₂ alkanols, C₃₋₂₂ alkane diols or C₃₋₂₂ alkane triols, such as dicaprylyl carbonate (Cetiol® CC), or the esters according to the teaching of DE 19756454 A1, and in particular glycerol carbonate.

Further cosmetic oils that may be preferred are selected from the esters of dimers of unsaturated C₁₂ to C₂₄ fatty acids (dimer fatty acids) comprising monohydric linear, branched or cyclic C₂ to C₁₈ alkanols or polyhydric linear or branched C₂ to C₆ alkanols.

Further cosmetic oils that are suitable are selected from silicone oils, which include, for example, dialkyl and alkyaryl siloxanes, such as cyclopentasiloxane, cyclohexasiloxane, dimethylpolysiloxane and methylphenylpolysiloxane, but also hexamethyldisiloxane, octamethyltrisiloxane and decamethyltetrasiloxane. Volatile silicone oils, which may be cyclic, can be preferred, such as octamethylcyclotetrasiloxane, decamethylcyclopentasiloxane and dodecamethylcyclohexasiloxane, and mixtures thereof, as they can be found in the commercial products DC 244, 245, 344 and 345 from Dow Corning, for example. Volatile linear silicone oils are likewise suitable, in particular hexamethyldisiloxane (L₂), octamethyltrisiloxane (L₃), decamethyltetrasiloxane (L₄), and arbitrary mixtures of two and three of L₂, L₃ and/or L₄, preferably mixtures such as those present, for example, in the commercial products DC 2-1184, Dow Corning® 200 (0.65 cSt) and Dow Corning® 200 (1.5 cSt) from Dow Corning. Preferred non-volatile silicone oils are selected from higher molecular weight linear dimethylpolysiloxanes, commercially available, for example, under the designation Dow Corning® 190, Dow Corning® 200 Fluid having kinematic viscosities (25° C.) in the range of 5 to 100 cSt, preferably 5 to 50 cSt, or 5 to 10 cSt, and dimethylpolysiloxane having a kinematic viscosity (25° C.) of approximately 350 cSt.

It may be exceptionally preferred to use mixtures of the aforementioned oils.

The agent can also include at least one protein hydrolysate and/or one of the derivatives thereof, for example, as a nourishing component. Protein hydrolysates are product mixtures that are obtained by the acidically, basically or enzymatically catalyzed degradation of proteins. The term “protein hydrolysates” shall also be understood to cover total hydrolysates and individual amino acids and the derivatives thereof, as well as mixtures of different amino acids. The molecular weight of the protein hydrolysates that can be used ranges between about 75, the molecular weight for glycine, and about 200,000; the molecular weight is preferably about 75 to about 50,000, and especially particularly preferably about 75 to about 20,000 daltons.

The agent can furthermore include at least one vitamin, a provitamin, a vitamin precursor and/or one of the derivatives thereof as a nourishing component. Vitamins, provitamins and vitamin precursors that are usually assigned to the groups A, B, C, E, F and H are preferred.

Further suitable nourishing components are in particular also linear fatty alcohols having 12 to 18 carbon atoms.

To set the pH, the cosmetic agent can comprise neutralizers or pH setting agents. Examples of neutralizers that are used in cosmetic agents are primary amino alcohols such as Aminomethyl Propanol (INCI), which is commercially available under the designation AMP-ULTRA® PC, for example, such as AMP-ULTRA® PC 2000.

The agents can furthermore comprise additional cosmetically acceptable preservatives. One example of a preservative that can preferably be used is 2-phenoxyethanol.

The cosmetic agent contemplated herein can be formulated in the forms customary for the temporary shaping of hair, for example as a wax, paste, lotion or clay. The cosmetic agents are preferably offered in jars or crucibles.

Another exemplary embodiment relates to the cosmetic, non-therapeutic use of cosmetic agents contemplated herein for temporarily shaping keratin fibers, and in particular human hair, and to a method for temporarily reshaping keratin fibers, and in particular human hair, in which the cosmetic agent is applied to keratin fibers.

A further exemplary embodiment relates to the use of o-phenol and/or salt(s) of o-phenylphenol in a cosmetic agent for temporarily reshaping keratin fibers, and in particular human hair, for reducing fading of the color of dyed keratin fibers. The dyed keratin fibers are preferably chemically dyed human hair. The cosmetic agent is preferably an agent as contemplated herein.

Tabular Overview

The compositions of several preferred cosmetic agents can be derived from the following tables (information as solids content and in percent by weight based on the total weight of the cosmetic agent, unless indicated otherwise).

| | Formula | Formula | Formula | Formula |
|---|---|---|---|---|
| | 1 | 2 | 3 | 4 |
| (a) Wax | 1-85 | 1.5-50 | 2-30 | 5-25 |
| (b) Emulsifier | 1-30 | 1.5-20 | 2-15 | 2-15 |
| (c) Polyhydric alcohol | 0.5-25 | 1-15 | 2-10 | 2-10 |
| (d) o-Phenylphenol | 0.01-0.25 | 0.05-0.225 | 0.1-0.2 | 0.1-0.2 |
| and/or-salt | | | | |
| (e) Water | 0.5-90 | 15-80 | 40-75 | 40-75 |
| Misc. | to make | to make | to make | to make |
| | up to 100 | up to 100 | up to 100 | up to 100 |
| | Formula | Formula | Formula | Formula |
| | 1a | 2a | 3a | 4a |
| (a) Wax | 1-85 | 1.5-50 | 2-30 | 5-25 |
| (b) Emulsifier | 1-30 | 1.5-20 | 2-15 | 2-15 |
| (c) Polyhydric alcohol | 0.5-25 | 1-15 | 2-10 | 2-10 |
| (d) o-Phenylphenol | 0.01-0.25 | 0.05-0.225 | 0.1-0.2 | 0.1-0.2 |
| and/or-salt | | | | |
| (e) Water | 0.5-90 | 15-80 | 40-75 | 40-75 |
| (f) Film-forming | 1-60 | 2-50 | 3-40 | 5-40 |
| polymer | | | | |
| Misc. | to make | to make | to make | to make |
| | up to 100 | up to 100 | up to 100 | up to 100 |
| | Formula | Formula | Formula | Formula |
| | 1b | 2b | 3b | 4b |
| (a) Plant-based wax | 1-85 | 1.5-50 | 2-30 | 5-25 |
| and/or microcrystalline | | | | |
| wax and/or petrolatum | | | | |
| and/or beeswax | | | | |
| (b) Emulsifier | 1-30 | 1.5-20 | 2-15 | 2-15 |
| (c) Polyhydric alcohol | 0.5-25 | 1-15 | 2-10 | 2-10 |
| (d) o-Phenylphenol | 0.01-0.25 | 0.05-0.225 | 0.1-0.2 | 0.1-0.2 |
| and/or-salt | | | | |
| (e) Water | 0.5-90 | 15-80 | 40-75 | 40-75 |
| (f) Vinylpyrrolidone- | 1-60 | 2-50 | 2-40 | 5-40 |
| containing polymer | | | | |
| Misc. | to make | to make | to make | to make |
| | up to 100 | up to 100 | up to 100 | up to 100 |
| | Formula | Formula | Formula | Formula |
| | 1c | 2c | 3c | 4c |
| (a) Beeswax and/or | 1-85 | 1.5-50 | 2-30 | 5-25 |
| petrolatum | | | | |
| (b) Emulsifier | 1-30 | 1.5-20 | 2-15 | 2-15 |
| (c) Polyhydric alcohol | 0.5-25 | 1-15 | 2-10 | 2-10 |
| (d) o-Phenylphenol | 0.01-0.25 | 0.05-0.225 | 0.1-0.2 | 0.1-0.2 |
| and/or-salt | | | | |
| (e) Water | 0.5-90 | 15-80 | 40-75 | 40-75 |
| (f) Vinylpyrrolidone- | 0 or 1-60 | 0 or 2-50 | 0 or 3-40 | 0 or 5-40 |
| containing polymer | | | | |
| Misc. | to make | to make | to make | to make |
| | up to 100 | up to 100 | up to 100 | up to 100 |
| | Formula | Formula | Formula | Formula |
| | 1d | 2d | 3d | 4d |
| (a) Beeswax and/or | 1-85 | 1.5-50 | 2-30 | 5-25 |
| petrolatum | | | | |
| (b) Addition products | 1-30 | 1.5-20 | 2-15 | 2-15 |
| of 2 to 100 moles | | | | |
| ethylene oxide to | | | | |
| linear fatty acids | | | | |
| having 12 to 18 | | | | |
| carbon atoms and/or | | | | |
| monoesters of fatty | | | | |
| acids having 12 to 18 | | | | |
| carbon atoms with | | | | |
| glycerol and/or | | | | |
| addition products of 2 | | | | |
| to 20 moles ethylene | | | | |
| oxide to beeswax | | | | |
| and/or phosphate | | | | |
| surfactants | | | | |
| (d) Polyhydric alcohol | 0.5-25 | 1-15 | 2-10 | 2-10 |
| (e) o-phenylphenol | 0.01-0.25 | 0.05-0.225 | 0.1-0.2 | 0.1-0.2 |
| (e) Water | 0.5-90 | 15-80 | 40-75 | 40-75 |
| (f) Vinylpyrrolidone- | 0 or 1-60 | 0 or 2-50 | 0 or 3-40 | 0 or 5-40 |
| containing polymer | | | | |
| Misc. | to make | to make | to make | to make |
| | up to 100 | up to 100 | up to 100 | up to 100 |
| | Formula | Formula | Formula | Formula |
| | 1e | 2e | 3e | 4e |
| (a) Plant-based wax | 1-85 | 1.5-50 | 2-30 | 5-25 |
| and/or microcrystalline | | | | |
| wax and/or petrolatum | | | | |
| and/or beeswax | | | | |
| (b) Addition products | 1-30 | 1.5-20 | 2-15 | 2-15 |
| of 2 to 100 moles | | | | |
| ethylene oxide to | | | | |
| linear fatty acids | | | | |
| having 12 to 18 | | | | |
| carbon atoms and/or | | | | |
| monoesters of fatty | | | | |
| acids having 12 to 18 | | | | |
| carbon atoms with | | | | |
| glycerol and/or | | | | |
| addition products of 2 | | | | |
| to 20 moles ethylene | | | | |
| oxide to beeswax | | | | |
| and/or phosphate | | | | |
| surfactants | | | | |
| (d) Polyhydric alcohol | 0.5-25 | 1-15 | 2-10 | 2-10 |
| (e) o-phenylphenol | 0.01-0.25 | 0.05-0.225 | 0.1-0.2 | 0.1-0.2 |
| (e) Water | 0.5-90 | 15-80 | 40-75 | 40-75 |
| (f) Vinylpyrrolidone- | 0 or 1-60 | 0 or 2-50 | 0 or 3-40 | 0 or 5-40 |
| containing polymer | | | | |
| Misc. | to make | to make | to make | to make |
| | up to 100 | up to 100 | up to 100 | up to 100 |

“Misc.” shall be understood to mean further customary components of cosmetic agents for temporarily shaping keratin fibers, such as perfumes/aromatic substances, polymeric thickening agents, pH-setting agents and/or nourishing components.

### EXAMPLES

The following cosmetic agents were produced:

| | | Ex- | Ex- | Ex- |
|---|---|---|---|---|
| | | ample | ample | ample |
| | | 1 | 2 | 3 |
| Component/ | | (% by | (% by | (% by |
| raw material | INCI name | weight) | weight) | weight) |
| Carnauba wax | Copernicia Cerifera | — | 0.1 | — |
| | Cera | | | |
| Beeswax | Beeswax | 2 | 0.5 | — |
| Petrolatum | Petrolatum | — | 82 | 2 |
| Microcrystalline | Cera Microcrystalina | — | 0.6 | — |
| wax | (Microcrystalline | | | |
| | Wax), Paraffin | | | |
| | Cetearyl Alcohol | 3 | — | — |
| | Stearyl Alcohol | — | — | 0.75 |
| Silicone Oil 50 CS | Dimethicone | 1 | — | — |
| D-Panthenol (75%) | Panthenol, Aqua | — | 0.7 | — |
| | Glycerin | 3 | — | — |
| Luviskol K 90 | PVP | 16 | — | 20 |
| (20%) | | | | |
| Antaron WP-660 | Triacontanyl PVP | — | — | 1.5 |
| Advantage LC-A | Vinyl Caprolactam/VP/ | — | — | 10 |
| | Dimethylaminoethyl | | | |
| | Methacrylate | | | |
| | Copolymer, Alcohol | | | |
| | Denat. | | | |
| PVP/VA | VP/VA Copolymer, | | | 10 |
| copolymer (50%) | Aqua | | | |
| StylezeCC-10 | VP/DMAPA | — | — | 10 |
| | Acrylates Copolymer | | | |
| 1,2-propylene | Propylene Glycol | — | — | 1.5 |
| glycol | | | | |
| 2-Phenylphenol | o-Phenylphenol | 0.2 | 0.1 | 0.15 |
| Aerosil R 812 S | Silica Silylate | 0.35 | — | — |
| Hostaphat KL | Trilaureth-4 Phosphate | — | 10 | — |
| 340 | | | | |
| Crodafos CES | Cetearyl Alcohol, | — | — | 1.5 |
| | Dicetyl Phosphate, | | | |
| | Ceteth-Phosphate | | | |
| Cutina CP | Cetylpalmitate | — | 3.8 | — |
| Ethoxylated stearic | Steareth-21 | — | — | 0.75 |
| acid (21EO) | | | | |
| | PEG-8 Beeswax | — | — | 0.3 |
| | Cetyl Alcohol | — | — | 0.75 |
| Arlacel 165 V P | Glyceryl Stearate, | 3 | — | — |
| | PEG-100 Stearate | | | |
| 2-amino-2- | Aminomethyl | 0.05 | — | — |
| methylpropanol | Propanol | | | |
| Tylose H | Hydroxyethylcellulose | — | — | 0.3 |
| 2-Phenoxyethanol | Phenoxyethanol | 0.5 | 0.48 | — |
| Nicotinamide | Niacinamide | — | 0.05 | — |
| L-Arginine | Arginine | — | 0.001 | — |
| EDTA Na2 | Disodium EDTA | — | — | 0.05 |
| Amaze | Corn Starch Modified | — | — | 0.5 |
| | DMDM Hydantoin | — | — | 0.3 |
| Perfume | Perfume (Fragrance) | 0.35 | 1.5 | 0.35 |
| Water | Aqua (Water) | to | to | to |
| | | make | make | make |
| | | 100 | 100 | 100 |

The quantity information in the tables is provided in % by weight of the respective raw material, based on the total agent.

The cosmetic agents 1 to 3 were physically and microbiologically stable over a period of 12 weeks at various temperatures (room temperature, 0° C., 45° C., −10° C.). When the cosmetic agents 1 to 3 are used in chemically dyed hair, the hair exhibits a lower tendency toward fading than with agent not comprising o-phenylphenol.

All produced cosmetic agents exhibited outstanding application and distribution properties in the hair and showed no residue on the treated hair.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. A cosmetic agent for temporarily shaping keratin fibers, comprising:
(a) at least one wax having a melting point above about 37° C. in a total amount of about 1 to about 85 wt. %;
(b) at least one emulsifier in a total amount of about 1 to about 30 wt. %;
(c) at least one polyhydric alcohol in a total amount of about 0.5 to about 25 wt. %;
(d) o-phenylphenol and/or salt(s) of o-phenylphenol in a total amount of about 0.01 to about 0.25 wt. %; and
(e) water in a total amount of about 0.5 to about 90 wt. %,
wherein the weight information is based in each case on the total weight of the cosmetic agent.

2. The cosmetic agent according to claim 1, furthermore comprising (f) at least one film-forming polymer in a total amount of about 1 to about 60 wt. %.

3. The cosmetic agent according to claim 1, wherein the at least one wax (a) is selected from plant-based waxes, Petrolatum (INCI), beeswax (INCI: Beeswax), microcrystalline wax, and mixtures thereof.

4. The cosmetic agent according to claim 1, wherein the at least one wax (a) is a mixture of carnauba wax (INCI: Copernicia Cerifera Cera), Petrolatum (INCI), beeswax (INCI: Beeswax) and microcrystalline wax.

5. The cosmetic agent according to claim 1, wherein the at least one emulsifier (b) is selected from the group consisting of addition products of about 2 to about 100 moles ethylene oxide to linear fatty acids having 12 to 18 carbon atoms, monoesters of fatty acids having 12 to 18 carbon atoms with glycerol, addition products of about 2 to about 20 moles ethylene oxide to beeswax, phosphate surfactants, and mixtures thereof.

6. The cosmetic agent according to claim 2, wherein the at least one film-forming polymer (f) comprises vinylpyrrolidone as a monomer.

7. The cosmetic agent according to claim 6, wherein the at least one film-forming polymer is particularly preferably selected from the group consisting of polyvinylpyrrolidone, vinylpyrrolidone/vinyl acetate copolymer, Vinyl Caprolactam/VP/Dimethylaminoethyl Methacrylate Copolymer (INCI), VP/DMAPA Acrylates Copolymer (INCI), Triacontanyl PVP (INCI) and mixtures thereof.

8. The cosmetic agent according to claim 1, furthermore comprising at least one nourishing component.

9. The cosmetic agent according to claim 1, wherein the cosmetic agent is in the form of a hair wax, paste, lotion or clay.

10. (canceled)

11. A method for temporarily shaping keratin fibers, the method comprising the steps of:
providing a cosmetic agent comprising:
(a) at least one wax having a melting point above about 37° C. in a total amount of about 1 to about 85 wt. %;
(b) at least one emulsifier in a total amount of about 1 to about 30 wt. %;
(c) at least one polyhydric alcohol in a total amount of about 0.5 to about 25 wt. %;
(d) o-phenylphenol and/or salt(s) of o-phenylphenol in a total amount of about 0.01 to about 0.25 wt. %; and
(e) water in a total amount of about 0.5 to about 90 wt. %,
wherein the weight information is based in each case on the total weight of the cosmetic agent, and
applying the cosmetic agent to the keratin fibers.

12. The method according to claim 11, wherein providing comprises providing the cosmetic agent furthermore comprising (f) at least one film-forming polymer in a total amount of about 1 to about 60 wt. %.

13. The method according to claim 11, wherein providing comprises providing the cosmetic agent wherein the at least one wax (a) is selected from plant-based waxes, Petrolatum (INCI), beeswax (INCI: Beeswax), microcrystalline wax, and mixtures thereof.

14. The method according to claim 11, wherein providing comprises providing the cosmetic agent wherein the at least one wax (a) is a mixture of carnauba wax (INCI: Copernicia Cerifera Cera), Petrolatum (INCI), beeswax (INCI: Beeswax) and microcrystalline wax.

15. The method according to claim 11, wherein providing comprises providing the cosmetic agent wherein the at least one emulsifier (b) is selected from the group consisting of addition products of about 2 to about 100 moles ethylene oxide to linear fatty acids having 12 to 18 carbon atoms, monoesters of fatty acids having 12 to 18 carbon atoms with glycerol, addition products of about 2 to about 20 moles ethylene oxide to beeswax, phosphate surfactants, and mixtures thereof.

16. The method according to claim 12, wherein providing comprises providing the cosmetic agent wherein the at least one film-forming polymer (f) comprises vinylpyrrolidone as a monomer.

17. The method according to claim 16, wherein providing comprises providing the cosmetic agent wherein the at least one film-forming polymer is particularly preferably selected from the group consisting of polyvinylpyrrolidone, vinylpyrrolidone/vinyl acetate copolymer, Vinyl Caprolactam/VP/Dimethylaminoethyl Methacrylate Copolymer (INCI), VP/DMAPA Acrylates Copolymer (INCI), Triacontanyl PVP (INCI) and mixtures thereof.

18. The method according to claim 11, wherein providing comprises providing the cosmetic agent furthermore comprising at least one nourishing component.

19. The method according to claim 11, wherein providing comprises providing the cosmetic agent in the form of a hair wax, paste, lotion or clay.
